(19) United States
(12) Patent Application Publication
(10) Pub. No.: US 2022/0392000 A1
(43) Pub. Date: Dec. 8, 2022

(54) SYSTEM FOR SUPPLYING TAILOR MADE SAFETY FACEMASKS AND METHOD THEREOF

(71) Applicant: Mats Moosberg, Torslanda (SE)

(72) Inventor: Mats Moosberg, Torslanda (SE)

(73) Assignee: Gear Perfect AB, Torslanda (SE)

(21) Appl. No.: 17/338,744

(22) Filed: Jun. 4, 2021

(51) Int. Cl. G06Q 50/04; A41D 13/11; A62B 18/00; A62B 23/02; G06Q 10/08

(52) U.S. Cl. CPC G06Q 50/04; A41D 13/11; A62B 18/003; A62B 23/025; G06Q 10/08

(57) ABSTRACT

The present invention relates to a system for supplying tailor-made safety facemask kit or a tailor made mask liner. The facemask kit includes a moldable strip liner. The moldable strip liner can be molded easily by heating at certain temperatures. The heating means can be either by microwave, induction heating, or electromagnetic field. The moldable strip liner can be supplied either separately or can be inserted/attached to the face mask. The tailor made safety facemask comprises a transparent pre-cut sheet, an upper mask liner and a nose-part. The facemask parts are customized and manufactured to fit single individual face geometry or a group of individual face geometries. In several other embodiments of the present invention, the upper mask liner can be made up of heat moldable materials, which can easily formed by heating below 100 C. A method of covering a face by using a tailor made mask liner is also disclosed.

### CROSS REFERENCE TO RELATED APPLICATIONS

This application is a non-provisional application and claims the benefit under 35 U.S.C. 119 (e) of U.S. provisional application No. 63/035,070 filed on Jun. 5, 2020 and hereby incorporated by reference in their entireties into this application

### FEDERALLY SPONSORED RESEARCH AND DEVELOPMENT

Not applicable.

### MICROFICHE

Not applicable

### (1) FIELD OF THE INVENTION

The present invention generally relates to the field of protective facemasks. The invention, particularly relates to a tailor made facemask kit or parts thereof. This invention, further relates to a system of supplying or delivering a tailor made facemask kit or parts thereof and method thereof.

### (2) BACKGROUND OF THE INVENTION

A respirator is a device designed to protect the wearer from inhaling hazardous or irritating atmospheres, including fumes, vapours, extreme temperatures, gases and particulate matter such as dusts and airborne microorganisms. There are two main categories: the air-purifying respirator, in which reparable air is obtained by filtering a contaminated atmosphere, and the air-supplied respirator, in which an alternate supply of breathable air is delivered. Within each category, different techniques are employed to reduce or eliminate noxious airborne contaminants. Air-purifying respirators range from relatively inexpensive single-use, disposable face masks sometimes referred to as a dust mask to more robust reusable models with replaceable cartridges often called a gas mask.

Many different types and styles of face-masks have been advanced, and each in its own fashion is suitable for a particular purpose. The faces of people vary greatly with respect facial features regarding size, arrangement, facial contours, etc. which makes it difficult for people to fit a pre-molded mask. Consequently, a need still exists for an inexpensive face-mask that can be used for long periods of time and will fit the special facial features of the individual wearer.

A number of different types of safety facemask and manufacturing of facemasks are available in prior art. For example, the following patents are provided for their supportive teachings and are all incorporated by reference: U.S. Pat. No. 9,381,316 discloses a mask assembly for a user includes a frame having a main body and lateral connector portions and a cushion component provided to the frame and defining a breathing cavity configured to accommodate at least a portion of the user's nose in use. The cushion component includes a face contacting seal portion adapted to seemingly engage with at least a portion of the user's nose in use. The cushion component has an aperture to communicate pressurized gas from the breathing chamber to the user's airways in use, and a main wall portion, opposite from the aperture, extending upwardly away from the frame. The main wall portion includes a stiffening portion. Another prior art document, CN105930880A 3D printing medical mask, which comprises a face side contacting with a face and an instrument side contacting with an instrument. The face side is characterized by obtaining personalized data for three-dimensional reconstruction through face scanning, and generating a face-shape-fit face side 3D data file; the instrument side is characterized by carrying out scanning on a conventional mask to obtain data for three-dimensional reconstruction, or carrying out correction according to clinical requirements to obtain a 3D data file; by jointing the 3D data files of the face side and the instrument side, a 3D printing mask digital file is formed; and finally, a personalized 3D printing medical mask is generated through printing of a 3D printer. However, this prior art document does not appear to discuss safety facemask kit comprising different parts of facemask.

Yet another prior art document, U.S. Pat. No. 6,098,201 discloses a face-mask which is made of a plastic sheet in which is embedded a deformable skin-colored, plastic-coated wire mesh. The plastic sheet is moldable and has an octagonal shape that will fit over the face of a wearer to cover the wearer's nose and mouth with tabs to fit under the chin. A trapezoidal opening is formed in the plastic sheet at a location that will overlie the nose and mouth of the wearer. A fastener system, located at the perimeter of the opening, surrounds the opening to fasten detachably a filter element in a position that covers the opening. The filter element is in sheet form having a shape complementary to the shape of the opening. However, this prior art document does not appear to discuss safety facemask kit comprising different parts of facemask.

Yet another prior art document, WO2014151324 discloses a face mask for extended wear by a user including a customized, contoured facial mask face surface portion constructed and configured to cover and to contact a corresponding contoured surface area of a human face, preferably formed by 3-D printing methods and materials. However, this prior art document does not appear to discuss safety facemask kit comprising different parts of facemask.

Yet another prior art document, US20080092909 discloses a medical face mask comprising a central transparent portion, an outer filter portion and, optionally, one or more antimicrobial agents. However, this prior art document does not appear to discuss a tailor made safety facemask.

Yet another prior art document, U.S. Pat. No. 9,962,511 discloses a shroud for a mask system includes a retaining portion structured to retain a frame, a pair of upper headgear connectors each including an elongated arm and a slot at the free end of the arm adapted to receive a headgear strap, and a pair of lower headgear connectors each adapted to attach to a headgear strap. The remaining portion, the upper headgear connectors, and the lower headgear connectors are integrally formed as a one piece structure. However, this prior art document does not appear to discuss a tailor made safety facemask or its kit.

Yet another prior art document, U.S. Ser. No. 10/424,113 discloses an apparatus and associated methods relate to fitting a virtual mask to a virtual face by first fitting a chin region of the virtual mask to the virtual face, then determining an virtual mask angle that maintains the fitted chin region while simultaneously fitting a nose-bridge region of the virtual mask to the virtual face, and then calculating a fit-quality metric corresponding to the fitted position. In an illustrative embodiment, the fitted chin region may include the high curvature mention region of the chin. In some examples, a virtual mask may be virtually pressed toward the virtual face using a predetermined force corresponding to a force of a mask securing device of a real mask corresponding to the virtual mask In an exemplary embodiment, the fitting of a virtual mask to a virtual face may advantageously yield a mask's fit quality in a brief amount of time. However, this prior art document does not appear to discuss a tailor made safety facemask kit or supplying of tailor-made safety facemask.

Yet another prior art document, US20080251210 discloses an automatic nose strip bonding apparatus for a face mask is disclosed, including a nose strip providing mechanism, a nose strip turnover mechanism, and a press-to-bond device. The nose strip providing mechanism drives a nose strip forward and carries out stamping operation on the nose strip, and applies a bonding agent to the nose strip. The nose strip turnover mechanism serves to turn over the nose strip that has already been applied with the bonding agent to allow the surface of the nose strip on which the bonding agent is applied to face a face mask. However, this prior art document does not appear to discuss a tailor made safety facemask kit or supplying of tailor-made safety facemask.

Yet another prior art documents, CN104643314 discloses a closing frame for improving closing performance of a respirator and a face mask. The closing frame comprises a closing surface 2 which is designed according to the face characteristics of special individual; the closing frame can be matched with and fit to the face; the closing frame is printed and produced through a 3D printer. The closing frame be used when wearing the respirator or the face mask and is able to reduce air leaking from the respirator and the face mask, and therefore, the closing performance of the respirator and the face mask can be improved. However, this prior art document does not appear to discuss safety facemask kit comprising different parts of facemask.

Yet another prior art document, U.S. Pat. No. 9,802,017 discloses a method of forming at least a portion of a mask that is structured to engage at least a portion of the face of a patient. The method can be generally stated as including employing at least a portion of a data set that is stored on a machine-readable storage medium and that is representative of the shape of at least a portion of the face in generating a configuration for a cushion element of the mask. The cushion element is structured to extend from a support of the mask and to have an engagement end opposite the support that corresponds with the shape of the at least portion of the face. The method can be generally stated as further including subjecting a workpiece to a formation operation to cause at least a portion of the workpiece to be formed into the cushion element having the engagement end. However, this prior art document does not appear to discuss safety facemask kit comprising different parts of facemask.

However, above mentioned references and many other similar references has one or more of the following shortcomings: (a) discussing general concept of the facemask; (b) discussing only 3D printing of safety masks; (c) disclosing only closing frame of face mask or respiratory mask; (d) not discussing customized or tailor-made face or respiratory mask; (e) not discussing the use of specifically designed program for tailor-made masks; (f) most of the references discussing only manufacturing of one mask only, none of the reference discusses continuous process of manufacturing of mask.

The present application addresses the above mentioned concerns and short comings with regard to providing a tailor-made safety facemask kit. Further, a system for supplying of the tailor-made safety facemask kit as a whole or in parts and method thereof is also disclosed. Note that in the present invention the descriptive wording tailor made is to describe a part that is customized and manufactured to fit a single individual face geometry or a group of individual face geometries.

### (3) SUMMARY OF THE INVENTION

In the view of the foregoing disadvantages inherent in the known types of safety facemask and manufacturing of facemasks now present in the prior art, the present invention provides a facemask comprising an adjustable nose fit for perfectly closing the nose gap. As such, the general purpose of the present invention, which will be described subsequently in greater detail, is to provide a system for supplying tailor-made safety facemask kit or parts thereof which has all the advantages of the prior art and none of the disadvantages.

The main objective of the present invention is to provide a system for supplying tailor-made safety facemask kit or parts thereof, wherein said system comprising: a user or a wearer with an object device; a mask creator app; a plurality of servers; a supplier department; a logistic department; and a delivery department; wherein said tailor-made safety facemask kit comprises a transparent sheet, an upper mask liner, a pre-cut chin support, and a snap-in arrangement, wherein said upper mask liner is attached to the said transparent sheet.

Another objective of the present invention is to provide the system, wherein said tailor-made safety facemask kit is used for making a tailor made safety facemask.

Yet another objective of the present invention is to provide the system, wherein said upper mask liner is removable.

Yet another objective of the present invention is to provide the system, wherein said transparent sheet further comprises a plurality of holes.

Yet another objective of the present invention is to provide the system, wherein said upper mask liner further comprises a right-side upper mask liner, a left-side upper mask liner, and a nose part.

Yet another objective of the present invention is to provide the system, wherein said upper mask liner further comprises a plurality of pins.

Yet another objective of the present invention is to provide the system, wherein said upper mask liner further comprises a plurality of air holes and means of flexible flaps.

Yet another objective of the present invention is to provide the system, wherein said pre-cut chin support comprises a simple filter media or a powered air flow module with a filter media.

Yet another objective of the present invention is to provide the system, wherein said powered air flow filter module comprises a blower fan, a compartment, and a replaceable filter media cartridge.

Yet another objective of the present invention is to provide a method for delivering said tailor made safety facemask kit or its part thereof.

Yet another objective of the present invention is to provide 1. A method for delivering a tailor-made safety facemask kit or parts thereof to a user by a system, wherein said system comprises a user with an object device; a mask creator app; a server; a supplier department; a logistic department; and a delivery department; wherein said method comprising the following steps: Taking images and videos by said user using said mask creator app, wherein said mask creator app is installed on said object device of said user; Sending collected a data to said server, wherein said data comprises images, videos, and combination thereof taken by said user in a previous step; Analyzing said data by said system using said mask creator app installed at said server, wherein said system analyzes said data using a computer algorithm; Providing a list of parts of said tailor-made safety facemask kit to said user by said system; Selecting of parts by said user from said list of parts of said tailor-made safety facemask kit and placing an order; Communicating said order to said supplier department; Verifying received said order with the said data provided by said system; Checking inventory and Preparing a list of to-be manufactured parts by said system; Sending and Manufacturing said list of to-be manufactured parts by a manufacturing unit; Packaging and transporting a final order to said user by said logistic department; and Delivering said final order to said order by said delivery department.

One of the main objective of the present invention is to provide a tailor made mask liner made up of heat moldable material, wherein said tailor made mask liner is moldable at the temperature ranging from 40° C. (degree centigrade) to 100° C. (degree centigrade), wherein said tailor made facemask liner used with a face cover or a face mask for covering a face.

Yet another objective of the present invention is to provide the tailor made mask liner, wherein said heat moldable material is a polymeric material.

Yet another objective of the present invention is to provide the tailor made mask liner, wherein said polymeric material is a thermoplastic material.

Yet another objective of the present invention is to provide the tailor made mask liner, wherein said tailor made mask liner comprises a deformation zone and a fastening zone.

Yet another objective of the present invention is to provide the tailor made mask liner, wherein said fastening zone comprises fastening arrangements such as glue type adhesive, Velcro, hook-and-loop type fastener, mechanical snapping function or similar attachment function.

Yet another objective of the present invention is to provide the tailor made mask liner, wherein said tailor made mask liner can be moldable either by placing it in a hot water or by heating by a heating means.

Yet another objective of the present invention is to provide the tailor made mask liner, wherein said tailor made mask liner comprises an integrated resistive conductor or a receptor.

Yet another objective of the present invention is to provide the tailor made mask liner, wherein said tailor made mask liner comprises a first compartment, a second compartment, and a barrier, wherein said first compartment and said compartment are separated by said barrier.

Yet another objective of the present invention is to provide the tailor made mask liner, wherein said first compartment is filled with a liquid polymer and said second compartment is filled with a liquid hardener or a curative.

Yet another objective of the present invention is to provide a method for delivering said tailor made safety mask liner.

### (4) BRIEF DESCRIPTION OF THE DRAWINGS

The invention will be better understood and objects other than those set forth above will become apparent when consideration is achieved to the following detailed description thereof. The accompanying drawings facilitate an understanding of the various embodiments of this invention. Such description makes reference to the annexed drawings wherein:

FIG. 1 depicts a schematic representation of the system in accordance with the present invention.

FIG. 2 illustrates by a flow chart an example of a method for delivering tailor made safety facemask kit or its parts to the user in accordance with the present invention

FIG. 3 illustrates a tailor made safety facemask according to one of the embodiment of the present invention.

FIG. 4 illustrates pre-fabricated upper mask liners of the safety facemask according to one of the embodiment of the present invention.

FIG. 5 illustrates 3D model generated by the mask creator app for selecting a tailor made safety facemask parts according to one of the embodiment of the present invention.

FIG. 6A represents how the tailor made safety facemask having filter media is worn by the user on the face according to one of the embodiment of the present invention.

FIG. 6B depicts the exploded view of the powered air flow filter media of the tailor made safety facemask according to one of the embodiment of the present invention.

FIG. 7 depicts the tailor made safety facemask according to one of the embodiment of the present invention.

FIG. 8 depicts the tailor made respiratory mask with full face protection according to one of the embodiment of the present invention.

FIG. 9 depicts the tailor made mask liner according to one of the embodiment of the present invention.

FIG. 10 depicts the tailor made mask liner having air holes according to one of the embodiment of the present invention.

FIG. 11 depicts the tailor made mask liner made up of heat moldable material according to one of the embodiment of the present invention.

FIG. 12 depicts the tailor made mask liner made up of heat moldable material according to one of the embodiment of the present invention.

FIG. 13 depicts the face model wearing a beforehand acquired face protection with the addition of the tailor made safety facemask parts according to one of the embodiments of the present invention.

FIG. 14 depicts the tailor made mask liner according to one of the embodiment of the present invention.

FIG. 15 depicts the face model wearing a face protection with the addition of the tailor made safety facemask parts according to one of the embodiments of the present invention.

FIG. 16 depicts the heat moldable strip liner according to one of the embodiment of the present invention.

FIG. 17 depicts the heat moldable strip liner having receptible according to one of the embodiment of the present invention.

FIG. 18 depicts the moldable liner having liquid polymer according to one of the embodiment of the present invention.

### (5) DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense.

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims and their equivalents.

The present invention is described in brief with reference to the accompanying drawings. Now, refer in more detail to the exemplary drawings for the purposes of illustrating non-limiting embodiments of the present invention.

As used herein, the term “comprising” and its derivatives including “comprises” and “comprise” include each of the stated integers or elements but does not exclude the inclusion of one or more further integers or elements.

As used herein, the singular forms “a”, “an”, and “the” include plural referents unless the context clearly dictates otherwise. For example, reference to “a device” encompasses a single device as well as two or more devices, and the like.

As used herein, the terms “for example”, “like”, “such as”, or “including” are meant to introduce examples that further clarify more general subject matter. Unless otherwise specified, these examples are provided only as an aid for understanding the applications illustrated in the present disclosure, and are not meant to be limiting in any fashion.

As used herein, the terms “may”, “can”, “could”, or “might” be included or have a characteristic, that particular component or feature is not required to be included or have the characteristic.

Exemplary embodiments will now be described more fully hereinafter with reference to the accompanying drawings, in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein.

Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

Thus, for example, it will be appreciated by those of ordinary skill in the art that the diagrams, schematics, illustrations, and the like represent conceptual views or processes illustrating systems and methods embodying this invention. The functions of the various elements shown in the figures may be provided through the use of dedicated hardware as well as hardware capable of executing associated software.

Similarly, any switches shown in the figures are conceptual only. Their function may be carried out through the operation of program logic, through dedicated logic, through the interaction of program control and dedicated logic, or even manually, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named element.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various terms as used herein are shown below. To the extent a term used in a claim is not defined below, it should be given the broadest definition and persons in the pertinent art have given that term as reflected in printed publications and issued patents at the time of filing.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

For simplicity and clarity of illustration, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments generally described herein.

Furthermore, this description is not to be considered as limiting the scope of the embodiments described herein in any way, but rather as merely describing the implementation of various embodiments as described.

Selection of respiratory protection for pollution hazards is typically based upon the airborne concentration of the substance that the wearer is exposed to, fit of the respirator and the hazard exposure limit of that substance. There are several types of masks today used, for example (i) Surgical Mask (or safety mask); (ii) Respiratory mask (respirators); (iii) Simple fabric masks; (iv) Homemade face coverings. The surgical mask (or safety mask) is used for preventing the large particles expelled by the wearer, such as spit or mucous, from entering the environment. If wearer coughs or sneezes, the surgical mask will help collect the larger particles expelled. The respiratory masks comprising of the filter cartridge. It filters particles from the air when properly fitted, helping reduce the number of particles or germs the wearer breathes in. Generally, it is ideal for achieving sufficient filtering of PM 2.5. The respiratory masks are secured tightly to the face, with 2 head straps and a nose adjustable clip over the nose to allow for a more custom fit. The simple fabric masks, such as cotton sewed masks, and homemade masks, such as a neck tube or wrapped fabric, are mostly effective for protecting from temperature differences and in some cases contaminated droplets. However, for all of these it is possible that the mask will not perfectly fit to the wearer, resulting in low level of efficiency and low level of comfort for the wearer. Currently, the whole world is passing through the critical situation of coronavirus pandemic. In the context of a pandemic, stopping the infection in both directions can be equally important in preventing a communicable disease from spreading. In the time of pandemic situation, there is a need for all types of protective respiratory mask ranging from high end filter cartridge face masks to homemade fabric masks. For all of these types of face protections the fit and sealing function will contribute highly to both the protection level and comfort of the mask. Further there is need to manufacture respiratory masks or parts for respiratory masks in bulk production. Moreover, the medical professionals and security professionals are in need of regular supply of respiratory masks. To provide the customized (tailor-made) respiratory masks to each individual is kind of an impossible task. There are many prior art exists (as discussed in the background of the invention section), which discusses a preparation of tailor-made mask using 3D-printing. However, all references discusses the production of mask by 3D-printing only on the experimental scale. Moreover, by using the 3D-printing of the current technology it is not possible to print transparent layer of the respiratory mask, which can be useful for the wearer either while attending the patients or while using.

A solution to this problem is achieved in the present patent application is achieved by providing a novel system for supplying tailor-made safety facemask kit or parts thereof to the user. The novel system of the present invention is enables to receive the facial contour digital data which by an algorithm and computer system is used for selecting the safety facemask parts from the set of safety facemask parts available in the system, or manually selecting the safety facemask parts from the set of safety facemask parts available in the system, and quickly delivering the tailor made safety facemask or parts for a tailor made mask to the user. The present invention also discloses a method of supplying a tailor made safety facemask in parts or as a whole to the user in a bulk quantity. Further, the system, in an alternate embodiment, also connected with the manufacturing unit, where it is possible to produce safety facemask parts by three-dimensional technology in bulk quantity quickly. Further, the present invention also discloses a method for incorporating an heat moldable material in the liner parts, to enable fine tuning of the fit and sealing function by the wearer. Further, the present invention also discloses a method for enabling the use of a transparent material as one part of the kit for improved visual appearance. The present invention is focused on providing a system for supplying tailor-made safety facemask kit or a tailor made mask liner. The tailor made safety facemask kit is very easy to prepare in the bulk quantity and supplied instantly wherever it is required. The tailor made safety facemask mask comprises several parts which are manufactured as per the demand by the user or the wearer. The system for supplying a tailor made safety facemask comprises a user with an object device; a mask creator app; a plurality of servers; a supplier department; a logistic department; and a delivery department. Further, the tailor-made safety facemask kit comprises a transparent sheet, an upper mask liner, a pre-cut chin support, and a snap-in arrangement, wherein said upper mask liner is attached to the said transparent sheet. The tailor-made safety facemask kit is used for making a tailor made safety facemask. The upper mask liner comprises a right-side upper mask liner, a left-side upper mask liner, and a nose part, and a plurality of pins. In some of the embodiments, the upper liner may also comprises a plurality of air holes and means of flexible flaps. The objective of the present invention is to provide a tailor made mask liner. The tailor made mask liner is made up of heat moldable material, wherein said tailor made mask liner is moldable at the temperature ranging from 40° C. to 100° C. The tailor made facemask liner used with a face cover or a face mask for covering a face. Different designing of the tailor made mask liners are also discussed below in different embodiments. The present invention is also discloses a method of covering a face by using a tailor made mask liner. The method comprises the following steps: Receiving said tailor made mask liner by a user; Hearing said tailor made mask liner to between 40-100 degrees Celsius, in water or by other means of heating; Placing and Applying Pressure to deform said heated tailor made mask liner toward said user's face contour;

Cooling down deformed said tailor made mask liner; and Using said deformed tailor made mask liner with a home-made mask or a surgical mask.

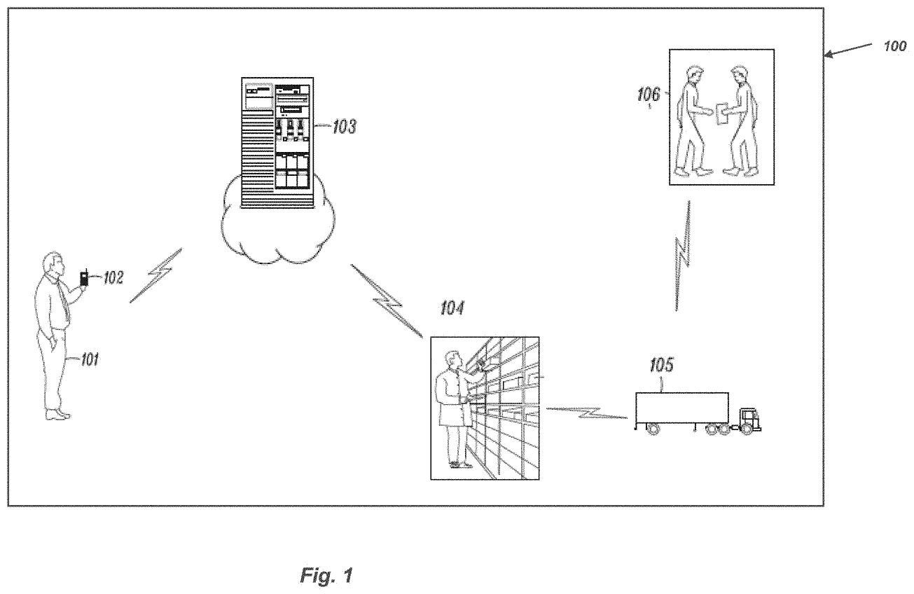

The main objective of the present invention is to provide the system for supplying a tailor made safety facemask as a complete kit, or fully assembled, or by supplying only the tailor made parts. In one embodiment the supplied parts can be fitted to an beforehand acquired face mask or an homemade fabric mask to improve performance of already available protective masks or surgical masks, or other means of facial protection. FIG. 1 shows schematic view illustrating one of the embodiments of this invention. The system 100 for supplying tailor made safety facemask comprising a user 101 with an object device 102, a mask creator app, a plurality of servers 103, a supplier department 104, a logistic department 105, and a delivery department 106. The object device comprises smartphone, mobile phone, wearable device, wraparound device, hand held device, mobile station, vehicle computer system, PDA device, personal computer, computer, monitor, smart TVs, displays, laptop, notebook, internet pad, virtual reality glasses, or the similar mobile or fixed applications. The server 103 may include external server, internal server, or cloud storage. The supplier department 104 once received the order then checks the availability of the safety facemask parts in the store. If all safety facemask parts available in the store then prepares the order. In case, the ordered safety facemask parts are not available in the stock, then in an alternate arrangement, the supplier department connects the safety facemask manufacturing unit and provides the details of the order. The manufacturing unit has facility of manufacturing tailor made safety facemask parts in bulk quantity either by three-dimensional technology or by any other suitable technology.

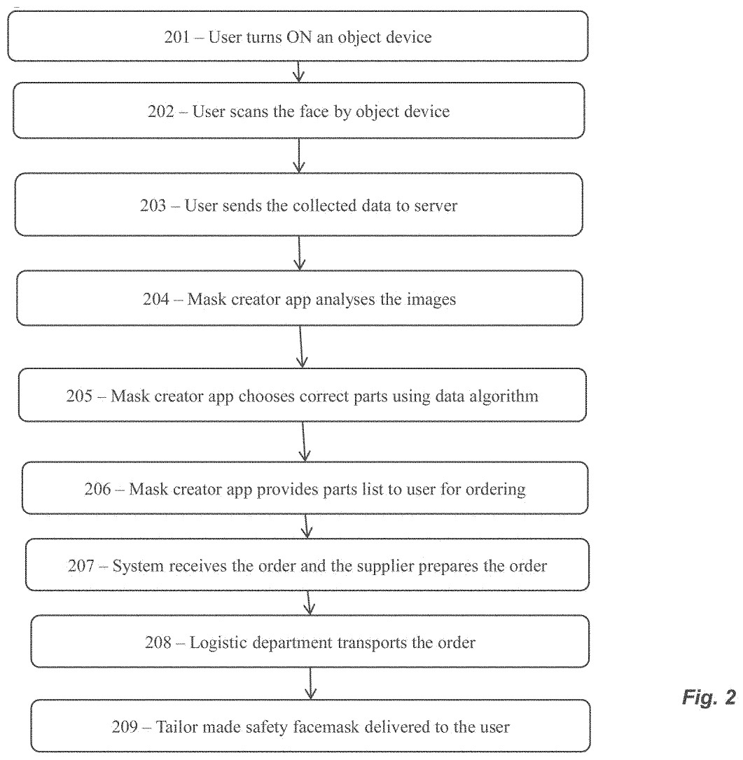

Another objective of the present invention is to provide the method of supplying a tailor made safety facemask in bulk quantity (mass quantity) quickly. The tailor-made facemask kit and its part can also be manufactured by the additive manufacturing process. FIG. 2 illustrates an example by a flow chart of supplying tailor made safety facemask kit in bulk quantity. The method includes the following steps: Step 201, the user turns ON the object device and starts the mask creator app. In next Step 202, the user scans the face by taking either various videos, still images or combinations of both. In next step 203, the user sends the collected images and/or videos to the server. The mask creator app can be either installed on the server. The mask creator app analyses the collected data (step 204). The mask creator app chooses different parts of safety facemask from the tailor made safety facemask data (model) using the specially designed algorithm (step 205). The mask creator app provides list of parts of tailor made safety facemask to the user for ordering (step 206). The user confirms the order on the system. The system then communicates the list of tailor made safety facemask parts to the supplier department (step 207). The system checks the inventory of the store and prepares the list of to-be manufactured parts. It is important to note here that the store has pre-fabricated different sizes of various parts of the safety facemasks. The various parts of the safety facemask can be transparent pre-cut sheets, right-side upper mask liners, left-side upper mask liners, nose-parts, chin-fitting parts, and filter cartridges. These set of parts of the safety facemask are manufactured by considering the various parameters, such as age of the users, facial structure/arrangement of the users, requirement of the users, etc. Further, the expert at the supplier department also verifies the list of tailor made safety facemask received from the user and the facemask data (model) for making sure to deliver the correct safety facemask to the user. In an alternate system arrangement, in case, the any of the ordered safety facemask part is not available with the store, then the supplier department sends out the request to manufacture the safety facemask part(s) to the manufacturing unit. The manufacturing unit has facility of manufacturing tailor made safety facemask parts in bulk quantity either by three-dimensional technology or by any other suitable technology. The manufacturing unit may also manufactured a tailor-made facemask kits and its parts by using an additive manufacturing technique.

In next step 208, then the logistic department transports the tailor made facemask kit to the user. The user receives the tailor made facemask kit from the delivery department (step 209). The user then assembles the various tailor made facemask parts. The supplied parts can also be intended for use with a beforehand available face protection. In an alternate method for supplying tailor made safety facemask, it is also possible that the production unit has the capabilities assembling the different tailor made parts of the facemask. The assembling can be done either by machine or by manually.

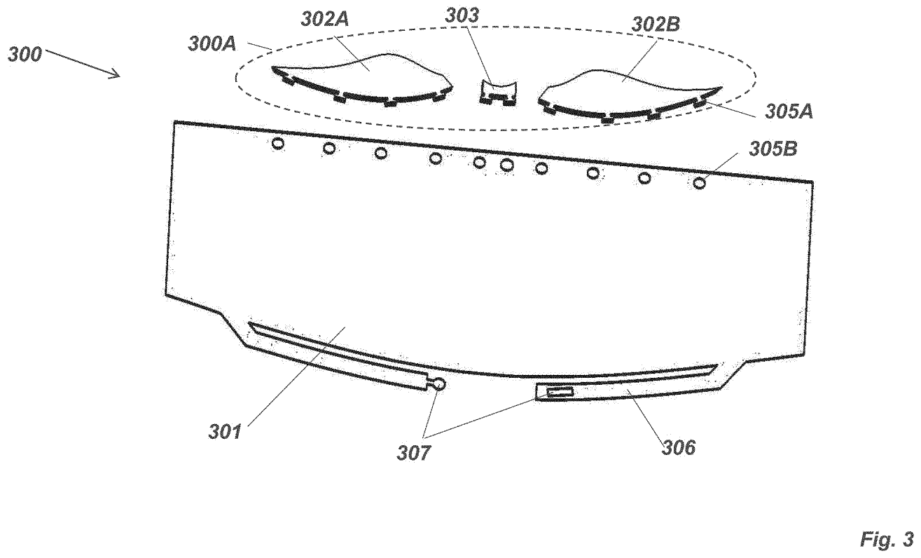

FIG. 3 illustrates a tailor made safety facemask according to one of the embodiment of the present invention. The tailor made safety facemask 300 comprises a transparent sheet (or a transparent pre-cut sheet) 301, an upper liner 300A, and a pre-cut chin support 306. The transparent sheet 301 comprises a plurality of holes 305B. The upper liner comprises a right-side upper mask liner 302A, a left-side upper mask liner 302B, and a nose-part 303. Further, the right-side upper mask liner 302A, the left-side upper mask liner 302B, and nose-part 303 also comprise a plurality of pins 305A. The plurality of pins 305A is designed in such a way that it can be fitted into the plurality of holes 305B of the transparent pre-cut sheet 301. The transparent pre-cut sheet 301 also has an arrangement at the bottom to pre-cut chin support 306. The pre-cut chin support 306 has an arrangement snap-in 307 to close chin region. In an alternate embodiment, a tailor made safety facemask may also comprise a filter cartridge (filter media). The filter cartridge (or filter media) can be fitted or snapped on the lower part of the transparent pre-cut part. The transparent pre-cut sheet may have hole at the bottom, further the hole can be of different shape such as ring, oval or square. The readily available filter cartridge can be fixated by a snap in lid.

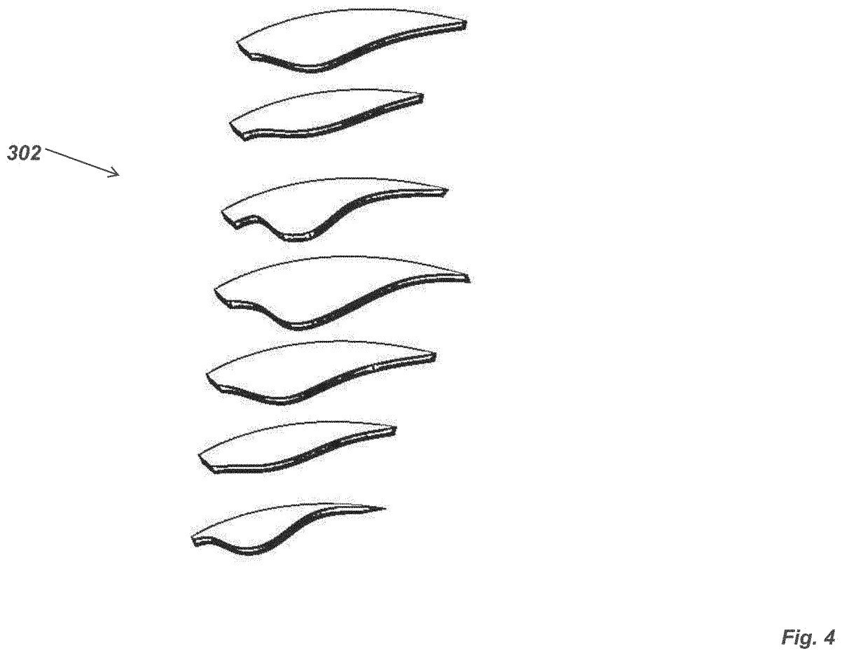

FIG. 4 illustrates pre-fabricated upper mask liners of the safety facemask according to one of the embodiment of the present invention. The number of pre-fabricated upper mask liners is represented in the FIG. 4. A number of different geometries are designed so that upper mask liner can achieve sufficient sealing function at the liner position for specific face geometry. The upper mask liner is manufactured by using a soft material, such as polyurethane, natural rubber, thermoplastic elastomer, foamed polymer, closed cell sponge material, silicone, etc. The soft material is used so there can be enough deformation in the liner to achieve sufficient sealing function with one specific liner part geometry for a large number of similar face geometries. In one embodiment the upper mask liner parts are manufactured partly or as a whole in a the heat moldable material, such as an EVA thermoplastic composition, PCL thermoplastic composition, TPU composition, PVA, PET, PLA or other thermoplastic composition with a heat distortion temperature below 100 C, to allow for final adjustment of fit by the user. By having a system of the present invention with a mask creator app (face scan application) and a server side system application, where the system can decide which prefabricated liner parts to use for a specific face geometry by using a geometry matching algorithm, or where the wearer manually selects the closest match from the prefabricated liner parts, where the liner parts are prefabricated and mass produced. The prefabricated tailor-made facemask kit and its part can also be manufactured by the additive manufacturing process. The pre-fabricated upper mask liners has enough deformation to achieve sufficient sealing function with one specific liner part geometry for a large number of similar face geometries, it will be possible to offer a comfortable fit respiratory mask with sufficient sealing function to a large number of a population.

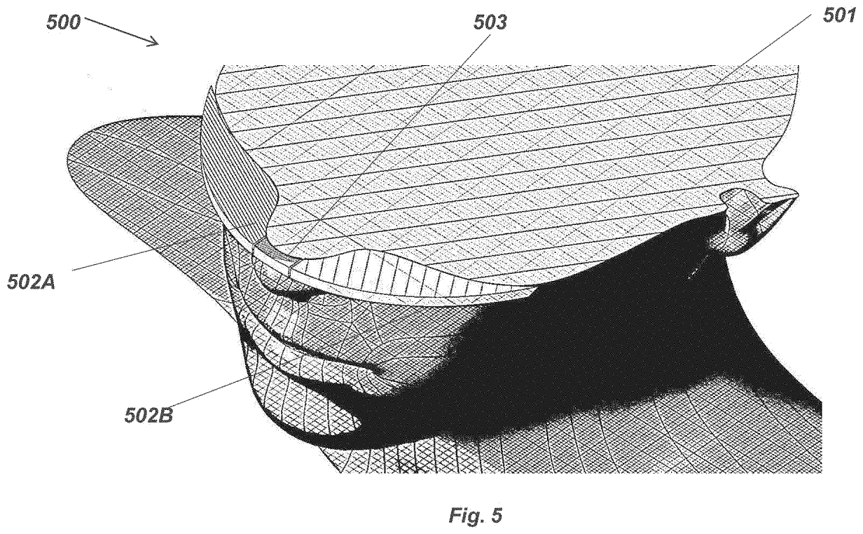

FIG. 5 illustrates 3D model and algorithm generated by the mask creator app for selecting a tailor made safety facemask parts according to one of the embodiment of the present invention. The mask creator app first creates the face structure 501 by analyzing the facial scan of the user. The mask creator app then calculates the distances and dimension require for a upper mask liner. The upper mask liner can be further fractioned to a right-side upper mask liner 502A, a left-side upper mask liner 502B, and a nose-part 503 by using a 2D section cut of the face structure, and by a line fit algorithm the closest matching prefabricated liner parts can be identified. FIG. 5 also illustrates the top view of the safety facemask.

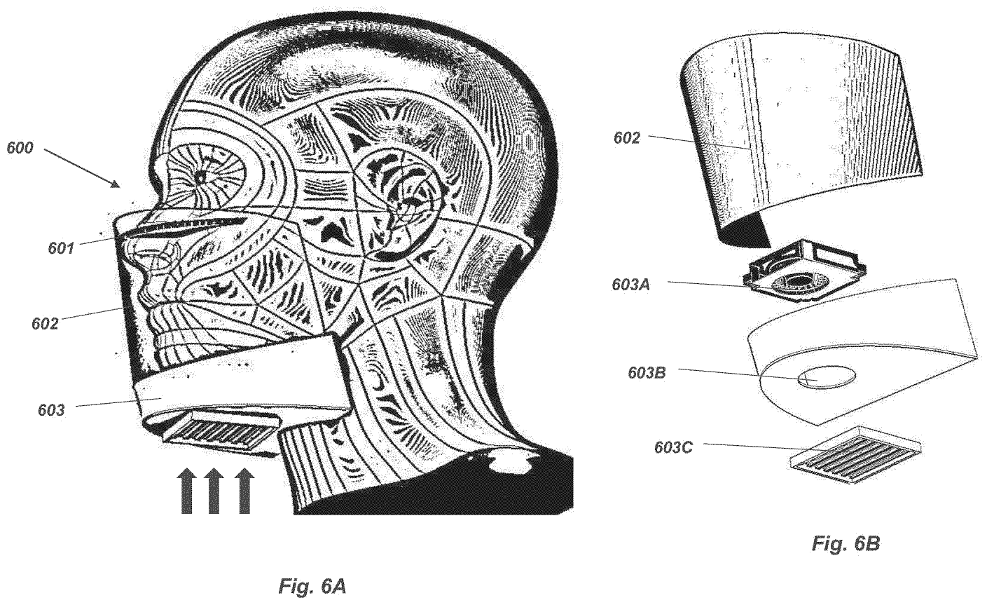

FIG. 6A and FIG. 6B depicts the tailor made safety facemask with forced air flow and a separable filter media according to one of the embodiment of the present invention. FIG. 6A represents how the tailor made safety facemask having filter media is worn by the user on the face. The tailor made safety facemask 600 comprises several parts, prefabricated upper mask liners 601, a transparent pre-cut sheet 602, and a powered air flow module with a filter media 603. The prefabricated upper mask liners 601 are selected based on the facial contour data received from the mask creator app. FIG. 6B depicts the exploded view of the powered air flow filter media. The powered air flow filter module 603 comprises blower fan 603A, compartment 603B, and replaceable filter media cartridge 603C. The compartment 603B can also be selected based on the scanned facial contour data received from the mask creator app. Further, the compartment 603B is designed to hold the blower fan 603A and replaceable filter media cartridge 603C. In this embodiment, the filter media cartridge includes a powered forced air flow to allow for a slight positive pressure in the mask inside cavity to increase comfort and easy of breathing. It can also reduce the moisture build up by providing a slight excess of outside air. All air entering the mask cavity will be filtered by a replaceable filter media part. The powered forced air can be controlled by a computer that monitors breathing and adjusts flow to relax the users breathing.

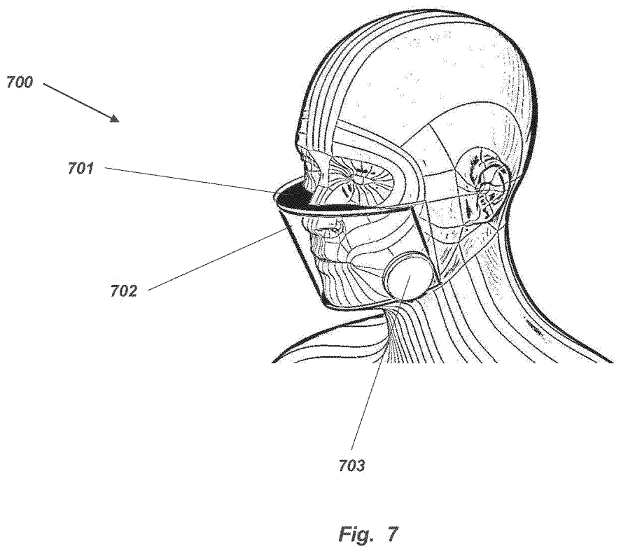

FIG. 7 depicts the tailor made safety facemask according to one of the embodiment of the present invention. The tailor-made safety facemask 700 is one of the embodiments, in which only mouth is protected. The tailor-made safety facemask 700 comprises mask liner (upper part) 701, transparent sheet 702, and filter cartridge 703. The filter cartridge 703 can be of round/circle shape. The filter cartridge can be fixated on the transparent sheet by snap-in, threaded attachment or other attachment type

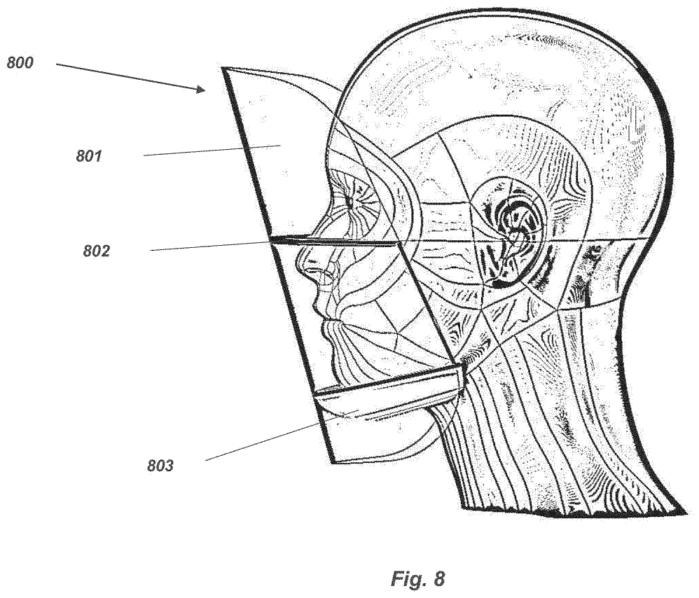

FIG. 8 depicts the tailor made safety facemask according to one of the embodiment of the present invention. The tailor-made respiratory mask 800 is one of the embodiments, which protects whole face including eyes. The tailor-made safety facemask 800 comprises upper mask liner 802, transparent sheet 801, and filter media 803. In this embodiment of the tailor made safety facemask, the transparent sheet is also extended to cover the eyes, to protect from droplets and splash. In one embodiment the transparent sheet is extended below the filter media to protect the filter media from droplets and splash.

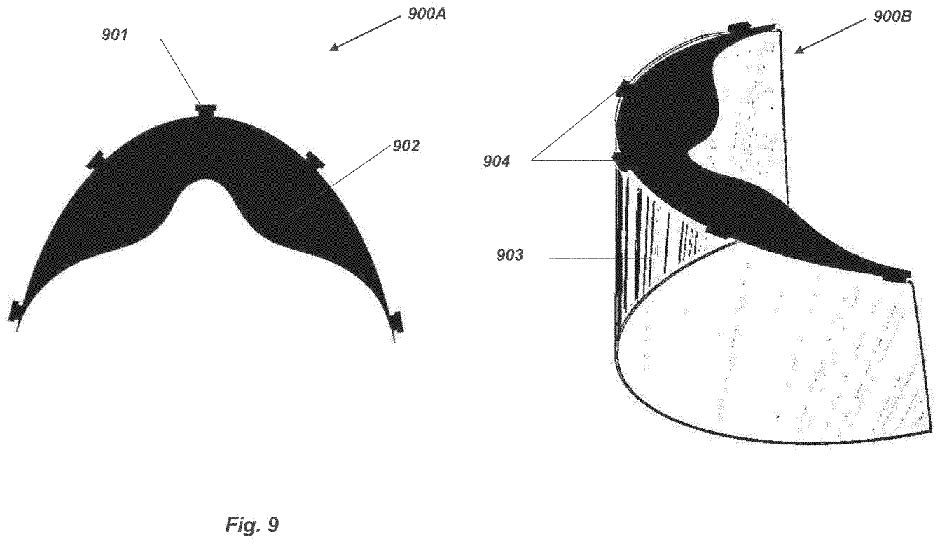

FIG. 9 depicts the tailor made safety facemask liner according to one of the embodiment of the present invention. The upper mask liner 900A and the transparent sheet is selected based on the collected data by the mask creator app. The mask creator app have chosen different parts of safety facemask from the tailor made safety facemask data (model) using the specially designed algorithm. The upper mask liner 900A comprises mask liner 902 and a plurality of pins 901. The transparent sheet 903 comprises a plurality of holes 904. The plurality of pins 901 are designed in such a way that it can be fitted on the plurality of holes 904 of the transparent sheet 903. The tailor made respiratory mask 900B is depicted in FIG. 9. In this embodiment, only the tailor made mask liner is manufactured by 3D-printing technology or other technology and which is fitted later on to the transparent sheet. The filter media not shown here will be attached to the lower part of the transparent sheet 903 or at other position on the transparent sheet. In one embodiment of the present invention the plurality of pins 901 are replaced by other means of fastening arrangements such as glue type adhesive, Velcro, hook-and-loop type fastener, mechanical snapping function or similar attachment function. In one embodiment of the present invention the precut transparent sheet 903 includes means for attachment function to the liner of mating type to the fastening arrangement 901 on the liner.

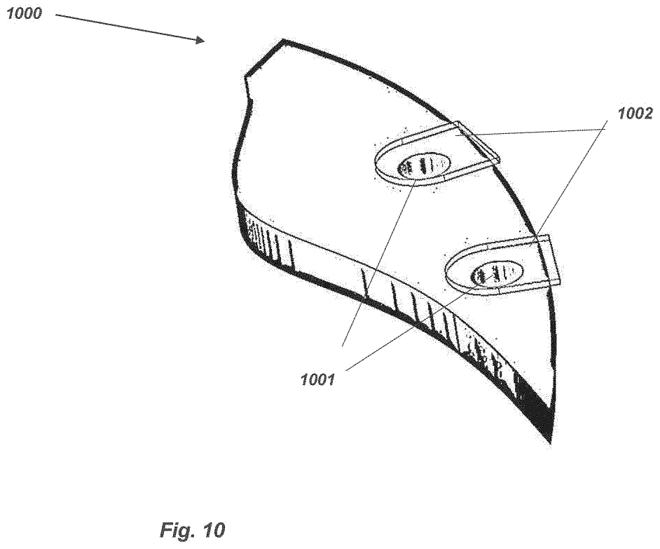

FIG. 10 depicts the tailor made mask liner having air holes according to one of the embodiment of the present invention. The upper mask liner 1000 and the transparent sheet is selected based on the collected data by the mask creator app. The mask creator app have chosen different parts of safety facemask from the tailor made safety facemask data (model) using the specially designed algorithm. The upper mask liner 1000 comprises plurality of air holes 1001 and means of flexible flaps 1002 for creating an one way valve to allow for exhaled air to pass directly to the outside, while still preventing inhaled air to pass to inside. This removes excess moisture from inside the mask. Further, the transparent sheet (which is not shown in the fig.) of the tailor made facemask can have an arrangement of the flexible flaps 1002. In this embodiment of the present invention these flexible flaps are integrated with the pre-cut transparent sheet and folded to meet the holes/air valves 1001 of the upper mask liner 1000. These flexible flaps are opening under over pressure. This will help wearer/user to breathe comfortably. The tailor made safety facemask kit or its part of the above mentioned different embodiments can be delivered by the method as depicted in the FIG. 2 flow chart, and which is also further explained earlier in the detail.

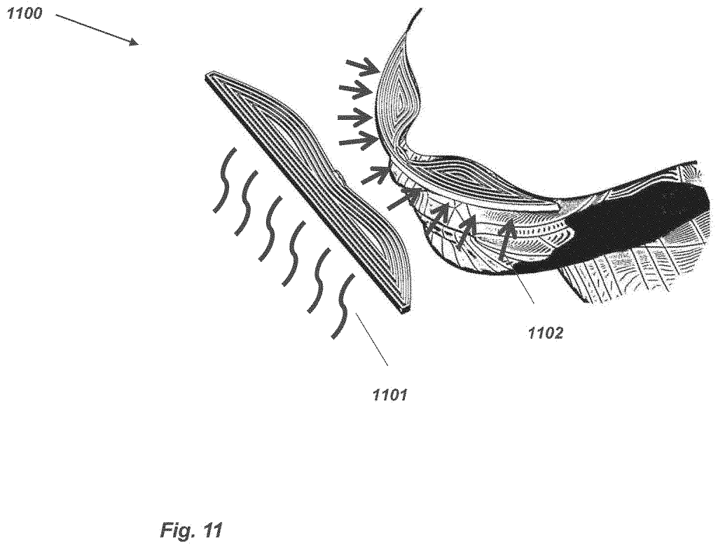

FIG. 11 depicts the tailor made mask liner according to one of the embodiment of the present invention. The objective of the present invention is to provide a tailor made mask liner. The tailor made mask liner is made up of heat moldable material, wherein said tailor made mask liner is moldable at the temperature ranging from 40° C. to 100° C. The tailor made facemask liner used with a face cover or a face mask for covering a face. The tailor made mask liner 1100 is made up of heat moldable material. The tailor made mask liner 1100 is manufactured partly or as a whole piece by using the heat moldable materials, such as an ethylene vinyl acetate (EVA) thermoplastic composition, Polycaprolactone (PCL) thermoplastic composition, Thermoplastic polyurethane (TPU) composition, PolyVinyl Alcohol (PVA), Polyethylene terephthalate (PET), Polylactic acid (PLA) or other thermoplastic composition with a heat distortion temperature below 100° C., to allow for final adjustment of fit by the user. The tailor made mask liner part will be supplied to the user. The user can heat the liner part to between 40-100 degrees Celsius, in water or by other means of heating 1101, and thereafter by applying pressure to the liner part toward the his/her face contour as illustrated with 1102 the tailor made mask liner part (upper liner part) will deform according to the specific face resulting in a close fit. This allows for supplier to supply the less number of variants needed to offer a comfortable fit respiratory mask with sufficient sealing function to a large number of a population. After cooling down it will remain in this deformation and can be attached to the pre-cut transparent sheet or to other type of protective mask cover.

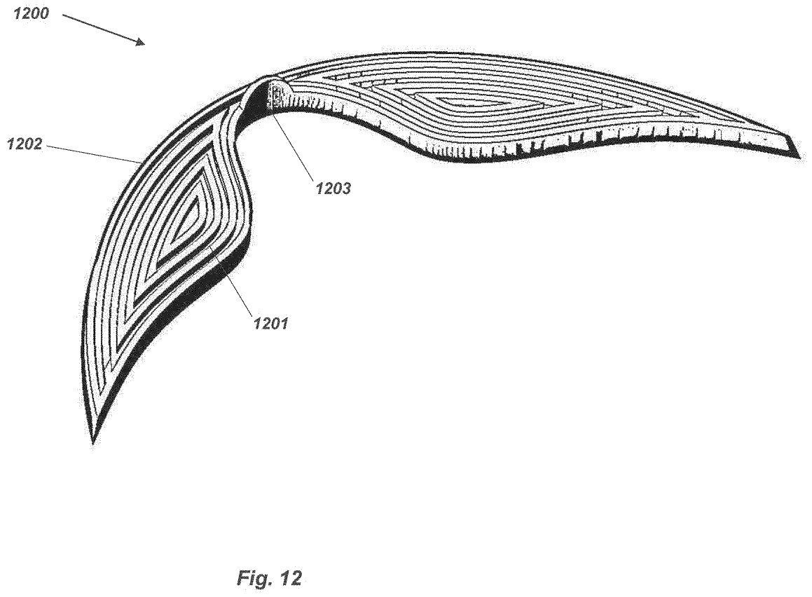

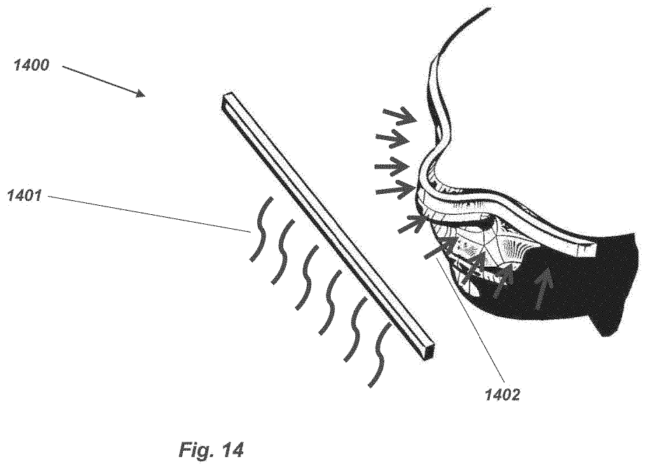

FIG. 12 depicts the tailor made mask liner according to one of the embodiment of the present invention. The tailor made mask liner 1200 is made up partly or as a whole of heat moldable material and it is same as discussed in FIGS. 11 and 14, however, the tailor made mask liner of this embodiment has two different region or zone. The tailor made mask liner 1200 has two zones, first is fastening zone 1202 and second is deformation zone 1201. The deformation zone 1201 is of folding type geometry for improved softness and deformability while heated. Further, the tailor made mask liner 1200 also has nose-support geometry 1203. The fastening zone 1202 has the fastening arrangements such as glue type adhesive, Velcro, hook-and-loop type fastener, mechanical snapping function or similar attachment function. The tailor made mask liner is attached to the pre-cut transparent sheet (or other available face protection parts) by this end/zone/area for tailored fit and good sealing and comfort level.

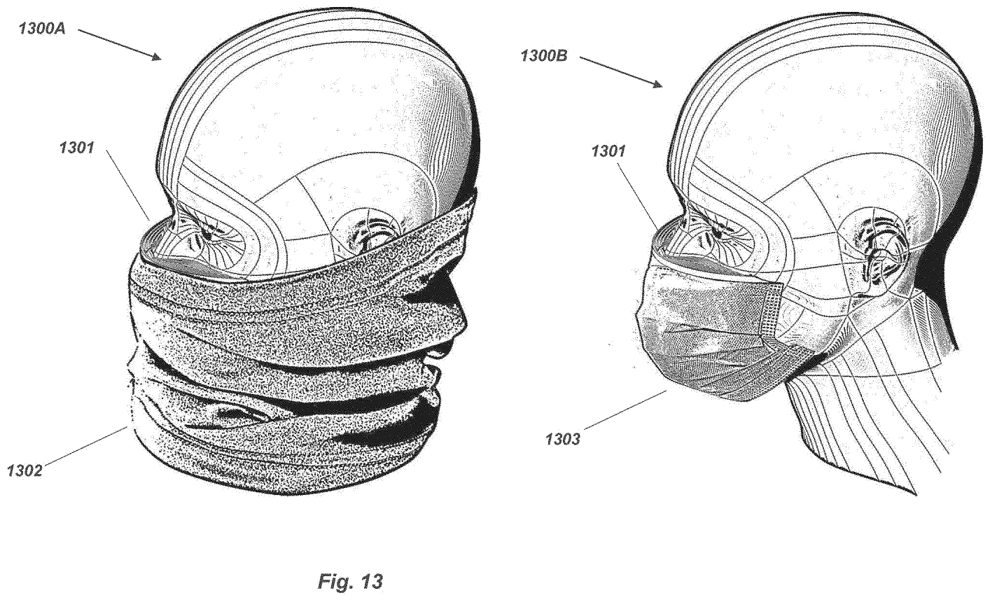

FIG. 13 depicts the face model wearing a beforehand acquired face protection with the addition of the tailor made safety facemask parts according to one of the embodiments of the present invention. The face model wearing the safety facemasks 1300A and 1300B are depicted in FIG. 13. The tailor made safety facemasks 1300A and 1300B comprises an upper mask liner 1301 as described in the present invention. The upper mask liner 1301 can be attached to a readily available face protection. The liner part(s) may include an attachment function such as glue type adhesive, velcro, hook-and-loop type fastener, mechanical snapping function or similar attachment function. The tailor made safety facemask 1300A shows the upper mask liner which is attached or connected or used with the home made mask 1302, which can be made out of cloth. While, the tailor made safety facemask 1300B depicts the use of upper mask liner 1301 of the present invention along with the readily available disposable surgical mask 1303.

FIG. 14 depicts the tailor made mask liner according to one of the embodiment of the present invention. The tailor made mask liner (thin liner strip) 1400 is made up of heat moldable material. The tailor made mask liner strip 1400 is manufactured partly or as a whole piece by using the heat moldable materials, with a heat distortion temperature below 100 C. This tailor made mask liner can be a heat moldable liner or a pre-fabricated liner as described in this application. In the case of a heat moldable liner the tailor made mask liner is straight, and heated and molded towards the face. In the case of a pre-fabricated liner the geometry is already correlating to the facial contour of the user at fabrication. The tailor made mask liner part will be supplied to the user. The user can heat the liner part to between 40-100 degrees Celsius, in water or by other means of heating 1401. The heat moldable strip is heated to the softening temperature of the chosen polymer, and then the surface of the strip needs to be cooled down to a temperature that is safe for skin, between 20-50 degrees Celsius, before applying to face.

After the strip is cooled down to a temperature that is safe for skin by applying pressure to the liner part toward the his/her face contour as illustrated with 1402 the tailor made mask liner part (upper liner part) will deform according to the specific face resulting in a close fit. After cooling down it will remain in this deformation and can be inserted into the face masks, in which there arrangement is given to place the tailor made mask liner 1400.

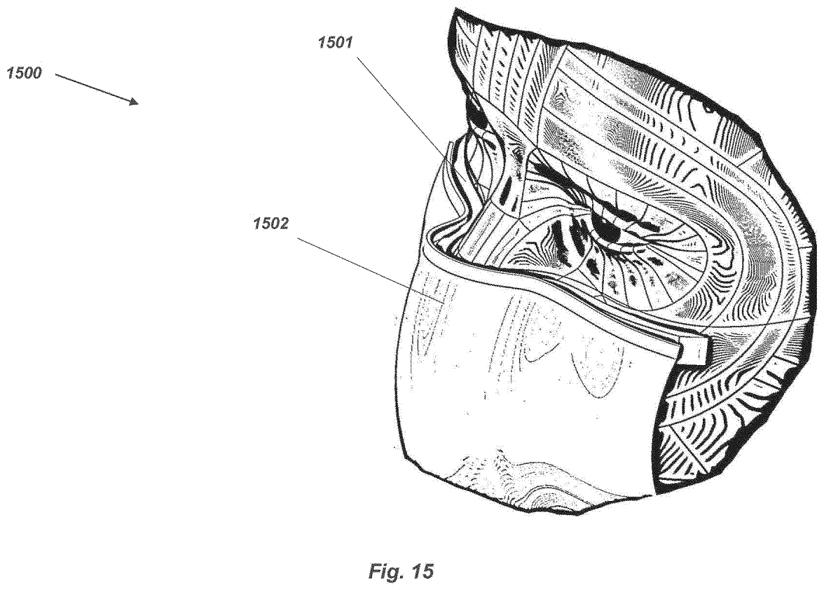

FIG. 15 depicts the face model wearing a face protection with the addition of the moldable tailor made mask liner according to one of the embodiments of the present invention. In one embodiment, tailor made mask liner (thin liner strip) 1501 can be attached to a face cover or face mask 1502, with an attachment means of a hook and loop type adhesive where the hook type strip is attached to the tailor made mask liner 1501 and the loop type strip is attached to the face cover 1502. In one embodiment, the tailor made mask liner 1501 can be attached to a face cover. The tailor made mask liner can be inserted in the face mask 1502. The face mask 1502 comprises a tube pocket. The tailor made mask liner can be included a sliding in tube pocket in the face mask 1502, where the tailor made mask liner 1501 can be inserted after molding. In one embodiment, tailor made mask liner 1501 can be attached to face mask 1502 by adding a glue type adhesive strip to the tailor made mask liner 1501 that adheres to the face mask 1502. Alternatively, the face cover (hand-made or a simple cloth type material) can also be used instead of the face mask. The tailor made mask liner 1501 is molded in the manners described in this patent application before attaching the tailor made mask liner 1501 to the face cover or face mask 1502.

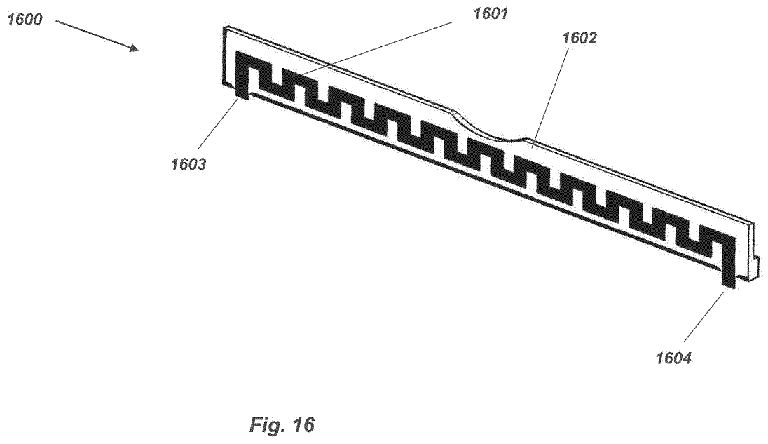

FIG. 16 depicts the heat moldable strip liner according to one of the embodiment of the present invention. The heat moldable tailor made mask liner (thin liner strip) 1600 is made up of heat moldable material. In one embodiment heat moldable strip liner 1600 has an integrated resistive conductor 1601 embedded in the heat moldable polymer material 1602. The resistive conductor liner can be connected to an external power supply at connector points 1603 and 1604 to heat up the liner to the polymer softening temperature. The temperature can be read by measuring the resistance of the integrated resistive conductor and a feedback loop can be implemented. When the heat moldable polymer is at the correct temperature (i.e., means mold temperature) the external power source can be disconnected and the liner can be pressed towards the facial contour of the user as described in this patent application. The heat moldable strip is heated to the softening temperature of the chosen polymer, and then the surface of the strip needs to be cooled down to a temperature that is safe for skin, between 20-50 degrees Celsius, before applying to face. In one embodiment the heat moldable strip liner is permanently attached to the face mask at production and the molding is done by the user, after the strip is cooled down to a temperature that is safe for the skin, with the complete mask to ensure best fit.

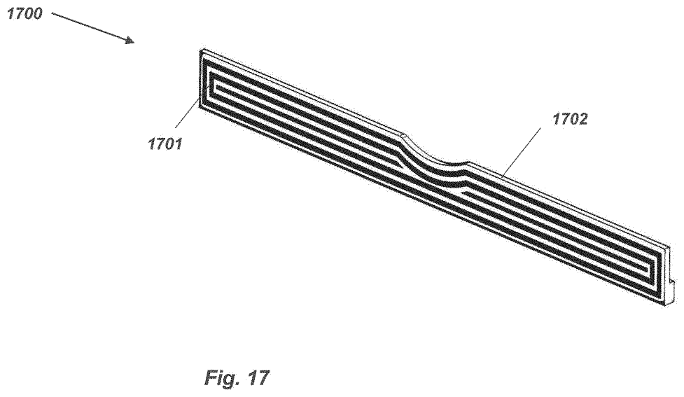

FIG. 17 depicts the heat moldable strip liner having receptible according to one of the embodiment of the present invention. In one embodiment heat moldable tailor made mask liner (thin liner strip) 1700 has a receptor 1701 embedded in the heat moldable polymer material 1702. The receptible is receptive of the induction heating or microwave heating. The receptor and polymer can be heated by an external source of a radio frequency alternating electric field or an alternating electromagnetic field. The temperature can be controlled by a time constraint on the heating, or by an external infrared thermometer feedback loop. In one embodiment the receptor material which is receptible to induction heating or microwave heating is embedded in the polymer in the shape of flakes or grains in the production process so to allow for a homogenous heating of the material. When the heat moldable polymer is at the correct temperature (i.e., means mold temperature) the external field can be turned off and the liner can be pressed towards the facial contour of the user as described in this patent application. The heat moldable strip is heated to the softening temperature of the chosen polymer, and then the surface of the strip needs to be cooled down to a temperature that is safe for skin, between 20-50 degrees Celsius, before applying to face. In one embodiment the heat moldable strip liner is permanently attached to the face mask at production and the molding is done by the user, after the strip is cooled down to a temperature that is safe for the skin, with the complete mask to ensure best fit.

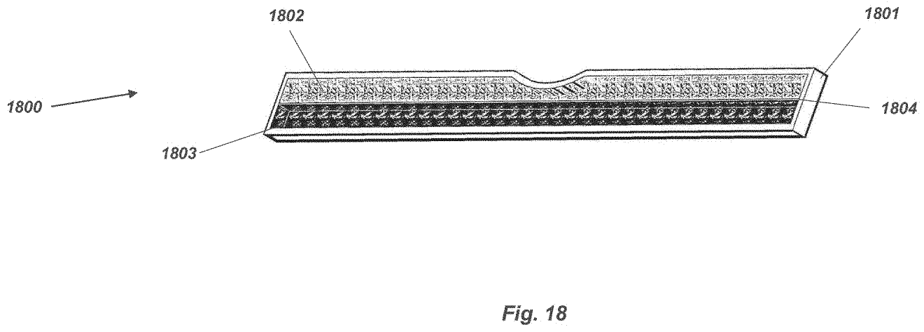

FIG. 18 depicts the moldable liner having liquid polymer according to one of the embodiment of the present invention. The moldable liner has liquid polymer and liquid hardener placed separately in two different compartment, which is separated by barrier. In one embodiment moldable liner 1800 is constructed in a soft flexible outside material 1801 and filled with a liquid polymer 1802 and a liquid hardener or curative 1803 separated by a barrier 1804. The user can by mechanical force break the barrier 1804 and initiate mixing of the two liquids and the curing/hardening process of the polymer. By applying the liner towards the users facial contour the polymer will harden to take on the contour of the users face. In one embodiment the moldable strip liner is permanently attached to the face mask at production and the molding is done by the user with the complete mask to ensure best fit. Further, in an alternate embodiment, the liquid polymer which is curable in the presence of ultra violet (UV) light can also be used. The moldable liner is constructed in such a way that the UV curable liquid polymer is placed in a compartment kind of arrangement. When, the user received the moldable liner, then he/she is heating the UV curable liquid polymer in the presence of UV light. By applying the liner towards the users facial contour the polymer will harden to take on the contour of the users face. In this embodiment the moldable strip liner is permanently attached to the face mask at production and the molding is done by the user with the complete mask to ensure best fit.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. A system for supplying tailor-made safety facemask kit or parts thereof, wherein said system comprising:
a user with an object device;
a mask creator app;
a plurality of servers;
a supplier department;
a logistic department; and
a delivery department;
wherein said tailor-made safety facemask kit comprises
a transparent sheet, an upper mask liner, a pre-cut chin support, and
a snap-in arrangement, wherein said upper mask liner is attached to the said transparent sheet.

2. The system according to claim 1, wherein said tailor-made safety facemask kit is used for making a tailor made safety facemask.

3. The system according to claim 1, wherein said upper mask liner is removable.

4. The system according to claim 1, wherein said transparent sheet further comprises a plurality of holes.

5. The system according to claim 1, wherein said upper mask liner further comprises a right-side upper mask liner, a left-side upper mask liner, and a nose part.

6. The system according to claim 1, wherein said upper mask liner further comprises a plurality of pins.

7. The system according to claim 1, wherein said upper mask liner further comprises a plurality of air holes and means of flexible flaps.

8. The system according to claim 1, wherein said pre-cut chin support comprises a simple filter media or a powered air flow module with a filter media.

9. The system according to claim 8, wherein said powered air flow filter module comprises a blower fan, a compartment, and a replaceable filter media cartridge.

10. A method for delivering said tailor made safety facemask kit or its part thereof of claim 1.

11. A method for delivering a tailor-made safety facemask kit or parts thereof to a user by a system, wherein said system comprises a user with an object device; a mask creator app; a server; a supplier department; a logistic department; and a delivery department;
wherein said method comprising the following steps:
Taking images and videos by said user using said mask creator app, wherein said mask creator app is installed on said object device of said user;
Sending collected a data to said server, wherein said data comprises images, videos, and combination thereof taken by said user in a previous step;
Analyzing said data by said system using said mask creator app installed at said server, wherein said system analyzes said data using a computer algorithm;
Providing a list of parts of said tailor-made safety facemask kit to said user by said system;
Selecting of parts by said user from said list of parts of said tailor-made safety facemask kit and placing an order;
Communicating said order to said supplier department;
Verifying received said order with the said data provided by said system;
Checking inventory and Preparing a list of to-be manufactured parts by said system;
Sending and Manufacturing said list of to-be manufactured parts by a manufacturing unit;
Packaging and transporting a final order to said user by said logistic department; and
Delivering said final order to said order by said delivery department.

12. A tailor made mask liner made up of heat moldable material, wherein said tailor made mask liner is moldable at the temperature ranging from 40° C. to 100° C., wherein said tailor made facemask liner used with a face cover or a face mask for covering a face.

13. The tailor made mask liner according to claim 12, wherein said heat moldable material is a polymeric material.

14. The tailor made mask liner according to claim 13, wherein said polymeric material is a thermoplastic material.

15. The tailor made mask liner according to claim 14, wherein thermoplastic material is selected from the an ethylene vinyl acetate (EVA) thermoplastic composition, Polycaprolactone (PCL) thermoplastic composition, Thermoplastic polyurethane (TPU) composition, PolyVinyl Alcohol (PVA), Polyethylene terephthalate (PET), Polylactic acid (PLA) or other similar thermoplastic composition.

16. The tailor made mask liner according to claim 12, wherein said tailor made mask liner comprises a deformation zone and a fastening zone.

17. The tailor made mask liner according to claim 16, wherein said fastening zone comprises fastening arrangements such as glue type adhesive, Velcro, hook-and-loop type fastener, mechanical snapping function or similar attachment function.

18. The tailor made mask liner according to claim 12, wherein said tailor made mask liner can be moldable either by placing it in a hot water or by heating by a heating means.

19. The tailor made mask liner according to claim 13, wherein said tailor made mask liner comprises an integrated resistive conductor or a receptor.

20. The tailor made mask liner according to claim 12, wherein said tailor made mask liner comprises a first compartment, a second compartment, and a barrier, wherein said first compartment and said compartment are separated by said barrier.

21. The tailor made mask liner according to claim 20, wherein said first compartment is filled with a liquid polymer and said second compartment is filled with a liquid hardener or a curative.

22. A method of covering a face by using a tailor made mask liner, wherein said method comprises the following steps:
Receiving said tailor made mask liner by a user;
Hearing said tailor made mask liner to between 40-100 degrees Celsius, in water or by other means of heating;
Placing and Applying Pressure to deform said heated tailor made mask liner toward said user's face contour;
Cooling down deformed said tailor made mask liner; and
Using said deformed tailor made mask liner with a home-made mask or a surgical mask.
